(19) United States
(12) Patent Application Publication
(10) Pub. No.: US 2024/0060897 A1
(43) Pub. Date: Feb. 22, 2024

(54) OPTOELECTRONIC CHIP

(71) Applicant: INTERHERENCE GMBH, Erlangen (DE)

(72) Inventors: Pierre Türschmann, Erlangen (DE); Daniel Böning, Erlangen (DE)

(21) Appl. No.: 18/270,157

(22) PCT Filed: Sep. 30, 2021
(86) PCT No.: PCT/EP2021/076977

§ 371 Date: Jun. 28, 2023

(30) Foreign Application Priority Data
May 11, 2021 (DE) 10 2021 112 251.1
May 11, 2021 (DE) 10 2021 112 256.2
Dec. 30, 2021 (DE) 10/2020 135 024.4

(51) Int. Cl. G01N 21/64; G02B 21/06; G02B 21/30; G02B 21/16; G01N 33/557

(52) U.S. Cl. CPC G01N 21/6458; G02B 21/06; G02B 21/30; G02B 21/16; G01N 21/6428; G01N 33/557; G01N 2201/0231; G01N 2201/08

(57) ABSTRACT

The present invention relates to an optoelectronic chip for receiving a sample in the visualization of temperature-dependent processes, having a carrier layer, a thin-film lightguide and a thin-film heating element, wherein the thin-film lightguide and the thin-film heating element are preferably arranged on sides of the carrier layer that lie opposite each other.

The present invention relates to an optoelectronic chip for receiving a sample in the visualization of temperature-sensitive processes and an optical system having such a chip, in particular a microscope for total internal reflection microscopy (TIRM).

TIR microscopy is preferably used to examine structures that are very close (between 0 and 200 nm for visible light) to a surface, for example a surface of an object carrier, in particular an optoelectronic chip, that is in contact with a sample. These can be, for example, fluorescently labelled molecules or scattering centers in or close to the membrane of a cell, individual DNA molecules bound to the surface or other structures. In contrast to conventional microscopy, TIR microscopy offers the advantage of better signal resolution. This is caused by selective illumination of the region of the cover glass close to the surface in conventional TIR microscopy or, in particular, in the region of the optical-electronic chip close to the surface. This illumination is produced by a field that decreases exponentially in intensity from the surface, also called an evanescent field. This generates a high contrast between the signal near the surface and the background scattered light.

Conventionally, TIR microscopy uses a lens with a very high numerical aperture to ensure that the light for optical excitation of the sample is totally reflected at an angle shallower than the critical angle at the interface between the cover glass and the sample. The illumination geometry, in particular the exponential drop of the evanescent field, is directly related to the angle at which the light exits the lens. However, the systems for setting this angle are highly temperature-sensitive and the field of observation is limited to a few hundred μm². As soon as the temperature of the sample and thus often also of the lens is changed by only a few degrees Celsius, the sample illumination changes significantly.

However, as TIR microscopy is often used to study temperature-sensitive biological processes (for example, to determine the binding affinity between a protein and an antibody or of living cells), it is essential to set the sample to a certain temperature to obtain usable data in many applications.

Against this background, an object of the present invention is to mitigate or even completely eliminate the problems of the prior art. In particular, the object of the present invention is to provide a device that eliminates the need for a lens with a very high numerical aperture and allows the sample to be set to a desired temperature reliably and quickly, as well as allowing larger observation fields of up to several mm² to be observed.

This object is achieved by an optoelectronic chip having the features of claim 1 and an optical system having the features of claim 13. Advantageous further developments of the present invention are the subject matter of the sub-claims.

An optoelectronic chip according to the invention is used for receiving a sample in the visualization of temperature-dependent processes and can thus be regarded as an object carrier.

Such an optoelectronic chip has a carrier layer, a lightguide (hereinafter also referred to as waveguide), preferably a thin-film lightguide, and a heating element, preferably a thin-film heating element, wherein the lightguide and the heating element are preferably arranged on opposite sides of the carrier layer.

Where the term thin-film lightguide is used in the following, it is to be understood that this reflects only a preferred embodiment and that other lightguides are also encompassed by the invention. Where the term thin-film heating element is used in the following, it is to be understood that this reflects only a preferred embodiment and that other heating elements are also encompassed by the invention.

The heating element and/or the lightguide is/are preferably optically transparent.

Optically transparent material is here preferably rather permeable to light in the range visible to humans, wherein the transmission of light through the optically transparent material is preferably at least 0.5, in particular at least 0.8. Optically opaque material is here preferably rather impermeable to light in the range visible to humans, wherein the transmission of light through the optically opaque material is preferably at most 0.49, in particular at most 0.3.

The lightguide and/or the heating element can be arranged directly on a surface of the carrier layer or be spaced apart from it via one or more intermediate layers.

In addition, the lightguide and/or the heating element and/or the carrier layer can each be designed as a single layer or as a composite of two or more sub-layers.

Preferably, the carrier layer consists completely or at least partially of an opaque or transparent material, preferably of Si or another SiO₂-based glass or crystal.

The carrier layer thus consists, for example, of glass, in particular borosilicate glass, and is preferably designed to give the optoelectronic chip mechanical stability.

Furthermore, between the carrier layer and the thin-film waveguide there can be a further transparent layer that has a lower refractive index than the carrier layer, preferably a refractive index between 1.2 and 1.5.

According to one embodiment of the invention, the carrier layer is made entirely or at least partially of a semiconductor material, preferably Si, and preferably a transparent layer, in particular a separating layer, is further present between the carrier layer and the lightguide, preferably the thin-film lightguide.

Furthermore, the thin-film heating element is preferably connected to and/or equipped with a temperature sensor, preferably in the form of a thin-film temperature sensor, which preferably forms at least a partial region of a surface of the optoelectronic chip, which is designed to come into direct or indirect contact with a sample.

For example, within the temperature sensor, a sensor layer for detecting the temperature of the sample may be provided, which preferably has metal and/or is made of metal and which preferably at least partially covers an outer surface of the optoelectronic chip and is further preferably designed to come into contact with a sample.

Preferably, the temperature is measured by the temperature sensor at at least one location in the sample, preferably at a plurality of locations to provide a more reliable reading.

Preferably, a four-wire measurement is used within the framework of the temperature sensor.

Furthermore, the optoelectronic chip preferably has a control unit to control and/or regulate the thin-film heating element on the basis of the measurement data regarding the sample temperature acquired by means of the temperature sensor.

Preferably, a thin-film heating element used in the invention is or comprises a resistance heating element. For example, carbon nanotubes can be used in the frame of the heating element.

In order to ensure that particles and/or objects and/or molecules to be examined can be localized close to a surface of the optoelectronic chip and thus in the range of the evanescent waves, it has proven advantageous in practice if an outer surface of the optoelectronic chip, which is designed to come into contact with a sample, at least partially or completely has a surface modification and/or surface functionalization in order to bind molecules (or other particles and/or objects) contained in the sample, in particular biological molecules.

For example, surface functionalization may involve providing the surface with certain functional chemical groups, such as hydroxy groups, to specifically bind a desired class of molecule to the surface.

The present invention further relates to a use of an optoelectronic chip according to the invention for receiving a sample in the visualization of temperature-dependent processes, wherein a sample, preferably an at least partially liquid, solid or gel-like sample is applied to the optoelectronic chip in such a way that the sample partially or completely covers the thin-film lightguide and preferably also the sensor layer of the temperature sensor. Furthermore, a chip according to the invention can be used with a microfluidic system.

For example, an optoelectronic chip according to the invention can be used to observe a temperature-sensitive process at a precisely controlled temperature of the sample. An optoelectronic chip according to the invention can further be used to examine the temperature dependence of a process by observing the process at different precisely controlled temperatures of the sample.

The sample used in the present invention preferably contains at least one or a plurality of particles and/or objects and/or molecules that are capable of and/or designed to interact with a guided mode (also referred to as mode) of the thin-film lightguide. For example, the molecules are excited to fluoresce by the light guided or conducted by the lightguide, deflect this light and/or absorb the light.

A further aspect of the invention relates to an optical system, preferably a microscope, particularly preferably a TIR microscope, which is adapted to be used with an optoelectronic chip according to the invention.

An optical system according to the invention preferably has at least one emitter that emits light for optical excitation into the thin-film waveguide and at least one detector that detects light deflected and/or emitted by the sample normal to the plane of the thin-film waveguide.

This design physically separates the light paths for excitation of the sample and detection of the light, reducing general scattered light that occurs when light is coupled into the waveguide or guided in the waveguide, and scattered light due to local scattering of light by the sample and background light. This leads to an improved ratio between the desired detected signals from the sample and unwanted signals caused by the measurement setup.

In order to guide light in a mode typical for the optical waveguide, coupling modules such as grating couplers, prism couplers and/or direct coupling mechanisms between two optical waveguides are preferably used. These coupling modules are used to introduce external light into the waveguide. More efficient coupling modules can increase the efficiency of the interaction between the emitter and the lightguide.

The lightguide directs the light of the excited guided mode over the optoelectronic chip and thus also through the volume of the sample.

The light guided by the lightguide can be reflected back and/or coupled out. For this purpose, coupling modules such as grating couplers, prism couplers and/or direct coupling mechanisms between two optical waveguides are preferably also used.

It is also conceivable that an optical mode of a different wavelength propagating simultaneously through the lightguide, different optical modes of the same wavelength or their combination are guided by means of additional coupling modules. An interaction of these within the waveguide and their detection can be used for highly sensitive measurements of the refractive index on the chip surface.

Preferably, in an optical system according to the invention, the detector is an array detector and/or the optical system is a microscope.

The invention further relates to a use of an optoelectronic chip according to the invention and/or of an optical system according to the invention for determining a phase transition of a particle (organic or inorganic) contained in the sample or of a spatially extended material. This phase transition may involve, for example, the modification of a biological molecule, such as an enzyme, a protein or a deoxyribonucleic acid (DNA) or ribonucleic acid (RNA).

Another aspect of the invention relates to a use of an optoelectronic chip according to the invention and/or an optical system according to the invention in the context of high-throughput sequencing, preferably based on single molecule analysis.

Yet another aspect of the invention relates to a use of an optoelectronic chip and/or optical system according to the invention for examining the binding affinities between at least one protein and at least one antibody as a function of temperature. The invention further relates to a use of an optoelectronic chip according to the invention and/or an optical system according to the invention for the examination of living cells under temperature-controlled conditions and their interactions with individual particles.

At this point it is pointed out that the terms “a” and “one” do not necessarily refer to exactly one of the elements, although this is a possible embodiment, but can also denote a plurality of the elements. Similarly, the use of the plural also includes the presence of the element in question in the singular and, conversely, the singular also includes several of the elements in question.

Further advantages, features and effects of the present invention are shown in the following description of preferred exemplary embodiments with reference to the corresponding figures. In the figures:

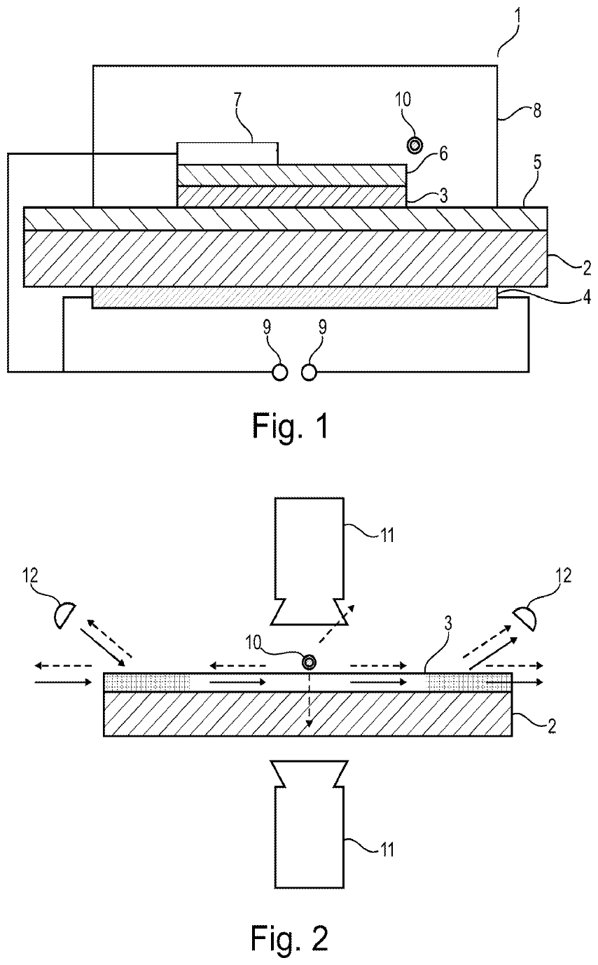

FIG. 1 schematically shows an optoelectronic chip according to the invention;

FIG. 2 schematically shows an optoelectronic system according to the invention;

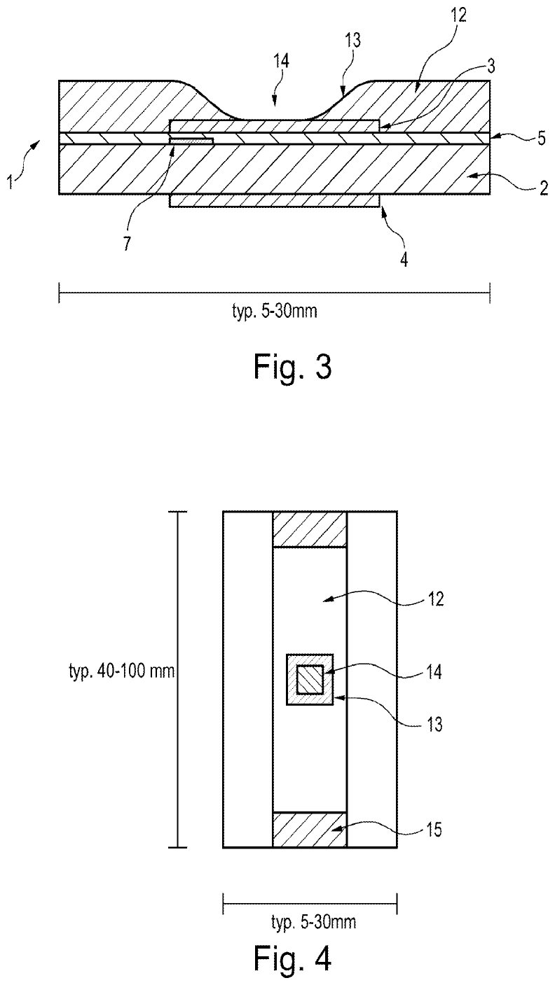

FIG. 3 shows a cross-section of another optoelectronic chip according to the invention; and

FIG. 4 shows a top view of the optoelectronic chip according to the invention from FIG. 3.

FIG. 1 shows an optoelectronic chip 1 according to the present invention. The chip 1 has a carrier layer 2, in this design made from a transparent silicon-based material, a thin-film lightguide 3 and a thin-film heating element 4. The thin-film lightguide 3 and the thin-film heating element 4 are arranged on two sides of the carrier layer 2, which lie opposite each other.

A separating layer 5 made from a transparent material is arranged between the carrier layer and the thin-film lightguide 3.

On the side of the lightguide 3 facing away from the carrier layer 2, a further separating layer 6 is arranged, which is positioned between the lightguide 3 and a metallic sensor layer 7, which is used to detect the temperature of the sample 8. In this embodiment, the sample 8 contains a particle 10 or also another object, for example a molecule.

In this embodiment, the sensor layer 7 is connected to a control unit (not shown), which controls and/or regulates the heating element 4 on the basis of the data detected by means of the sensor layer 7. The electronic circuit for controlling and/or regulating the heating element 4 is only shown in stylized form in FIG. 1 and is designated by the reference numeral 9.

The heating element 4 can be operated in a feedback mode in which the read-out value of the temperature sensor, in particular of the sensor layer 7, is used as a feedback parameter. Regulation preferably takes place electronically. It is also possible to use the heating element 4 unregulated or without feedback control.

FIG. 2 shows an optical system in which a chip 1 according to the invention is used. The chip 1 is shown very simplified in this representation; only the carrier layer 2 and the lightguide 3 are shown.

Light is coupled into the lightguide 3 from an emitter (not shown) to excite it. In the representation in FIG. 2, the light of the excited guided mode of the lightguide 3 propagates from left to right through the lightguide 3, as shown in FIG. 2 by the arrows with continuous lines.

The particle 10, which is very close to the surface of the lightguide 3, can interact with the light of the propagating guided mode of the lightguide 3, for example by the particle absorbing the light or being excited by the light to fluoresce. For example, the particle 10 can deflect, scatter and/or reflect the light, as shown in FIG. 2 by the arrows with dashed lines.

Light influenced and deflected in this way by the particle 10 can, for example, be detected by means of detectors 11, e.g. imaging systems. In this case, the path of the light thus detected preferably runs normal, i.e. perpendicular, to the plane of the thin-film lightguide 3 and thus also normal or perpendicular to the propagation direction of the excited guided mode of the lightguide 3.

The light path for exciting the lightguide 3 (from left to right in FIG. 2) and the light path for detecting light from the sample 8 or particle 10 (top and bottom in FIG. 2) are thus spatially separated from each other in this embodiment and preferably run normal, or in other words orthogonal, to each other.

In other words, spatially resolved detection of the light deflected by the sample at right angles to the plane of the lightguide 3 from above and/or from below through the carrier layer of the chip 1 thus takes place. The light deflected by the sample perpendicular to the plane of the lightguide 3 may be, for example, red-shifted or blue-shifted relative to the guided mode of the lightguide or resonant to it.

The sample and/or particles therein interact(s) with an evanescent wave originating from the guided mode of the lightguide, causing the guided mode light to be, for example, scattered, absorbed or re-emitted at a different wavelength.

Furthermore, photodetectors 12 can be used to detect the resonant light guided in the lightguide 3 in the guided mode and/or to detect the light scattered in the guided mode of the lightguide 3. The light scattered into the guided mode of the lightguide 3 is, for example, red-shifted or blue-shifted relative to the guided mode of the lightguide.

A typical procedure for optical excitation and detection, as used in the context of a system according to FIG. 2, is reproduced below:

Light is sent or introduced into the waveguide mode via a coupling module. A portion of the coupling light can be reflected, while another portion of the light can be transmitted through the mode. The transmitted light can be scattered again in a second coupling region.

Both parts of the light can be detected via a photodetector and used, for example, as a feedback signal or control parameter for intensity stabilization of the light component guided in the waveguide. For such an intensity stabilization, the light reflected in the coupling region and/or the light transmitted in the decoupling region of the waveguide mode can be used as a feedback signal to stabilize or change the intensity of the light in the waveguide in a controlled manner. For this purpose, a chip according to the invention may have a correspondingly configured controller.

Light can be coupled into the waveguide via more than one coupling module. Alternatively or additionally, light with a different polarization, wavelength, propagation direction, etc. can be coupled in simultaneously or sequentially. Light scattered over the coupling areas can also be used to analyze the sample volume.

A particle 10 (e.g. a biomolecule) can interact with the guided mode via the evanescent light component (active sample region). The light scattered by the particle (fluorescence and/or direct scattered light) can be detected with local resolution by one or two optical systems or detectors 11, which are preferably located above or below the optoelectronic chip. Light scattered by the particle 10 can also couple into the waveguide mode and be scattered via the coupling modules and thus detected.

FIG. 3 shows a further embodiment of the invention. In particular, FIG. 3 shows a cross-section of an optoelectronic chip 1 with an exemplary arrangement of layers and layer thicknesses. The active region 14 is defined by a protective layer 12, which in this embodiment ends adiabatically (in principle, non-adiabatic transitions are also conceivable). The adiabatic transfer of the protective layer 12 is indicated by the reference numeral 13. Only the active region 14 of the chip comes into contact with a sample volume.

The protective layer 12 has a lower refractive index than the waveguide 3; preferably the refractive index of the protective layer is in the range of 1.3 to 1.5 in the visible range.

The adiabatic transfer 13 from the protective region or a region of the protective layer 12 facing away from or spaced apart from the active region 14 to the active region 14 enables a mode transition without a generation of scattered light and/or a loss of light power independent of a refractive index of the sample volume.

Furthermore, the protective layer 12 prevents the occurrence of contamination in the coupling region and scattered light from any optional container or channel for holding a sample on or in the optoelectronic chip. In the arrangement shown in FIG. 3, the sample can simply be picked up in the trough formed by the active region 14.

The heating element 4 can be arranged on a side of the chip 1 opposite to the active region 14. Alternatively or additionally, a heating element 4 may be present between the separating layer 5 and the carrier layer 2. A chip can also be provided with a plurality of heating regions.

The temperature sensor or at least its sensor layer 7 can be arranged between the separating layer 5 and the carrier layer 2. In principle, the sensor or at least its sensor layer 7 can also be arranged at another location on the chip 1, but in this case, it must always be ensured that the temperature sensor and/or a component thereof is not located in the evanescent field of the waveguide 3.

The temperature sensor or sensor layer 7, the separating layer 5 and the protective layer 12 are optional. Typical layer thicknesses of the layers used in an optoelectronic chip according to the invention are shown below: Protective layer 12 (optional): 100-1000 nm, preferably 300-800 nm, waveguide layer 3: 50-1000 nm, preferably 75-250 nm, separating layer 5 (optional): 100-1000 nm, preferably 100-800 nm, carrier layer 2: 150-1000 μm, preferably 170-500 μm, heating element 4: 5-100 nm, preferably 10-50 nm. The chip 1 shown in FIG. 3 has a width (dimension from right to left in FIG. 3) of 5-30 mm.

FIG. 4 shows a top view of the optoelectronic chip from FIG. 3. The chip 1 shown in FIG. 4 has a width (dimension from right to left in FIG. 4) of 5-30 mm and a length (dimension from top to bottom in FIG. 4) of 40-100 nm. One or a plurality of coupling regions 15 allow light to be coupled into and out of the guided waveguide modes. In the embodiment shown in FIG. 4, two coupling regions 15 are shown at opposite ends of the chip 1.

The coupling regions 15 and the waveguide 3 are preferably covered by the protective layer 12 and only in the active region 14 is the waveguide layer 3 exposed and can come into direct contact with the sample volume. The waveguide layer 3 can be partially or completely chemically functionalized. The active region 14 preferably has an area of 0.01 mm²−25 mm² and the total area of the optoelectronic chip 1 is preferably 25 mm²−2000 mm².

The present invention offers significant technical advantages, in particular the following advantages:

The present invention enables a large optical field of observation to be viewed. In particular, by separating the excitation and detection paths, it becomes technically possible to optically excite a larger sample region or active region. Conventional TIR systems typically illuminate sample regions of a few 10-3 mm², whereas the present invention enables optical excitation of region up to a few mm². This opens up new possibilities to detect a large number of biomolecules in a highly parallelized way. In addition, the present invention enables more complex biological systems such as cells or cell clusters to be optically excited and observed.

In addition, an optoelectronic chip according to the invention offers homogeneous illumination of the sample region or active region compared to conventional TIR systems.

The signal-to-background ratio is also improved with an optoelectronic chip according to the invention. Such an optoelectronic chip offers a greatly reduced scattered light background compared to conventional TIR systems, as the exciting light field is not guided through the detection optics.

Furthermore, the present invention offers the advantage that the penetration depth of the evanescent field into the sample volume can be varied over a wide range by cleverly choosing the waveguide layer parameters and the wavelength of the light. This penetration depth can vary from a few 10 nm to several 100 nm, depending on the layer and sample properties and the wavelength of the light.

In addition, optical excitation via the waveguide mode eliminates the need for lenses with a high numerical aperture, as the necessary angle of incidence of the light is supported by the waveguide mode. In addition, the use of immersion medium is no longer necessary for achieving the conditions of total internal reflection, which greatly improves the user experience.

Furthermore, the temperature of the sample can be adjusted very quickly by heating the sample volume locally using a chip according to the invention. According to the invention, heating rates of up to 100° C./s are possible. As only small sample volumes are heated, the heat capacity is low and already the environment allows a rapid cooling of the sample to ambient temperature with cooling or heat dissipation rates of more than −20° C./s.

Conventional approaches to macroscopic temperature regulation entail various disadvantages, such as long equilibration times, thermal drifts, degraded optical imaging properties, etc., which are circumvented by the present invention.

In addition, the integration of a thin-film temperature sensor in the optoelectronic chip enables direct feedback control of the heating element. This ensures highly precise and dynamic temperature regulation of the sample volume.

The high sensitivity of the optoelectronic chip with regard to optical excitation and thermal changes also enables the calorimetric detection of phase transitions.

By integrating a highly sensitive excitation, a heating element and optionally a temperature sensor into the optoelectronic chip, the overall system can also be greatly reduced in size and complexity.

Since the optoelectronic chip has no moving elements, mechanical wear or vibration of the entire system is avoided and the mechanical stability of the system is optimized.

The design of the optoelectronic chip is still fundamentally compatible with microfluidic channels. In particular, the use of a protective layer with adiabatic transition to the active sample region ensures the functionality of the excitation system independent of the refractive index of the sample volume, if this is lower than the mode index of the waveguide, and/or of a potential sample container or channel located on or in the optoelectronic chip.

1. Optoelectronic chip for receiving a sample in the visualization of temperature-dependent processes, having a carrier layer, a thin-film lightguide and a thin-film heating element, wherein the thin-film lightguide and the thin-film heating element are arranged either on sides of the carrier layer that are opposite each other or on the same side of the carrier layer.

2. Optoelectronic chip according to claim 1, characterized in that the carrier layer consists completely or at least partially of an opaque or transparent material, preferably of Si or another SiO₂-based glass or crystal.

3. Optoelectronic chip according to claim 1 or 2, characterized in that between the carrier layer and the thin-film waveguide there is a further transparent layer that has a lower refractive index than the carrier layer, preferably a refractive index between 1.2 and 1.5.

4. Optoelectronic chip according to claim 3, characterized in that the further transparent layer consists of a polymer or an amorphous or crystalline material.

5. Optoelectronic chip according to any one of the preceding claims, characterized in that the thin-film heating element is equipped with a temperature sensor, preferably in the form of a thin-film temperature sensor, and preferably a control unit is also provided to control and/or regulate the thin-film heating element based on the measurement data acquired by means of the temperature sensor.

6. Optoelectronic chip according to any one of the preceding claims, characterized in that the heating element is optically transparent and/or consists of an indium-tin-oxide compound.

7. Optoelectronic chip according to any one of the preceding claims, characterized in that the thin-film heating element is or comprises a resistance heating element.

8. Optoelectronic chip according to any one of the preceding claims, characterized in that a sensor layer is further provided, which preferably has metal and/or consists of metal and which preferably at least partially covers an outer surface of the optoelectronic chip and is further preferably designed to come into thermal contact with a sample.

9. Optoelectronic chip according to claim 8, characterized in that a temperature regulation takes place by means of a feedback system between the sensor layer and the heating element.

10. Optoelectronic chip according to any one of the preceding claims, characterized in that an outer surface of the optoelectronic chip, which is designed to come into contact with a sample, at least partially or completely has a surface modification and/or surface functionalization in order to bind molecules contained in the sample, in particular biological molecules.

11. Use of an optoelectronic chip according to any one of the preceding claims for receiving a sample in the visualization of temperature-sensitive processes, wherein a sample, preferably an at least partially liquid, solid or gel-like sample is applied to the optoelectronic chip in such a way that the sample partially or completely surrounds the thin-film lightguide.

12. Use according to claim 11, characterized in that the sample contains at least one or a plurality of particle(s) that is/are capable of and/or designed to interact with a guided mode of the thin-film lightguide.

13. Optical system, preferably a microscope, which is designed to be used with an optoelectronic chip according to any one of the preceding claims, having at least one emitter or scatterer that emits light for optical excitation of the sample parallel to the plane of the thin-film lightguide and having at least one detector that detects light deflected by the sample normal to the plane of the thin-film lightguide.

14. Optical system according to claim 13, characterized in that the detector is an array detector and/or the optical system is a microscope.

15. Use of an optoelectronic chip according to any one of claims 1 to 10 and/or an optical system according to claim 13 or 14 for determining a melting point of a single particle contained in the sample, preferably a biological molecule, for example an enzyme, a protein or a deoxyribonucleic acid (DNA) or ribonucleic acid (RNA) or in the context of a high-throughput sequencing based on the analysis of single molecules or for the examination of the binding affinities between at least one protein and at least one antibody as a function of temperature or for the examination of living cells under temperature-controlled conditions.
